(19) United States
(12) Patent Application Publication
(10) Pub. No.: US 2013/0089912 A1
(43) Pub. Date: Apr. 11, 2013

(54) METHODS AND COMPOSITIONS FOR THE STABILIZATION OF BIOLOGIC MATERIAL

(71) Applicants: Gloria Elliott, Midland, NC (US); Douglas R. MacFarlane, Brighton (AU)

(72) Inventors: Gloria Elliott, Midland, NC (US); Douglas R. MacFarlane, Brighton (AU)

(21) Appl. No.: 13/645,745

(22) Filed: Oct. 5, 2012

Related U.S. Application Data
(60) Provisional application No. 61/543,997, filed on Oct. 6, 2011.

(51) Int. Cl. C12N 5/071; C12N 5/079; C12N 5/077; G01K 17/00; C12N 5/076; C12N 5/0793; C12N 5/075; C12N 7/00; C12N 5/0775

(52) U.S. Cl. 435/235.1; 435/325; 435/374; 435/395; 374/31; 374/E17.001

(57) ABSTRACT

A method of screening an organic salt for potential toxicity if used as a stabilizing agent for live cells or viruses, the method comprising: (a) providing a composition of unilamellar vesicles; then (b) contacting an organic salt to the unilamellar vesicles; and then (c) detecting a change in at least one thermal parameter of the unilamellar vesicles caused by the organic salt at said known concentration. A change in the at least one thermal parameter indicates the organic salt is potentially toxic for use as a stabilizing agent for live cells or viruses. Compositions of organic salts identified by such methods, along with methods of using the same in stabilizing live cells or viruses, are also described.

### RELATED APPLICATIONS

This application claims the benefit of U.S. Provisional Patent Application Ser. No. 61/543,997, filed Oct. 6, 2011, the disclosure of which is incorporated by reference herein in its entirety.

### STATEMENT OF GOVERNMENT SUPPORT

This invention was made with Government support under Grant Nos. R21EB00740401 and R01EB01362901 from the NIH. The US Government has certain rights to this invention.

### FIELD OF THE INVENTION

This application concerns methods and compositions useful for stabilizing biological materials such as cells and viruses.

### BACKGROUND OF THE INVENTION

Existing technologies for preservation of biological constructs are no longer adequate to meet the demands of the rapidly growing biological therapeutics and tissue engineering industries. Where small molecule drugs once dominated the pharmaceutical repertoire, stem cell therapies, engineered tissue equivalents, protein-based therapeutics, and gene therapies (siRNA, microRNA, retrovirals), collectively referred to here as ‘biologics’, have emerged as the medicines of the future. Many of these therapies are targeted towards the individual, rapidly leading to an era of ‘personalized medicine’ (Baust (2005)). This increased variety and complexity of bio-based medicines has resulted in higher costs to produce, store, and transport these therapies. In particular, cell-based therapeutics must be stored and transported at cryogenic temperatures to minimize adverse chemical reactions that occur when molecular mobility is not constrained. The cost of routinely replenishing liquid nitrogen together with the associated technical equipment required to ensure constant temperatures results in very high handling costs.

This renders some therapeutics uneconomic to deliver and, in many cases, impossible to deliver beyond a well-resourced supply network. A similar picture is emerging for cells and tissues that are being stored for present and future biomarker studies (see, e.g., Mills (2009)). As the deputy director for advanced technologies and strategic partnerships at the National Cancer Institute once stated, “We must do something about the biobanking issue or personalized medicine will be delayed by 30 or 40 years” (Aldridge (2005)). New paradigms are needed to reduce the costs and increase the capacity to safely deliver new and emerging biological therapeutics.

Recent trends in cell preservation science have focused to a large extent on preservation in a dry state as an alternative to cryogenic storage. The dry preservation approach involves removing water from the formulation to create a highly viscous, low mobility matrix (i.e. a glass) that prevents degradation reactions, but does not require refrigerated storage (see, e.g., Katkov et al. (2006)). Other approaches include solution modifiers to extend short-term liquid storage time (Sitaula et al. (2009)). In these scenarios, classic cryoprotective agents are of little utility due to chemical toxicity effects at ambient/near-ambient temperatures and/or low glass transition temperatures (Tg). Trehalose, sorbitol, and mannitol, can form glasses at ambient conditions, and also provide molecular protection during processing, but these traditional pharmaceutical excipients have met with only moderate success in cell preservation applications, largely due to the requirement of extremely low water content to achieve a suitable glass (see, e.g., Elliott et al. (2008)). To move beyond these incremental advances, new materials and alternative storage technologies are critically needed.

### SUMMARY OF THE INVENTION

A first aspect of the invention is a method of screening an organic salt for potential toxicity if used as a stabilizing agent for live cells or viruses, the method comprising: (a) providing a composition of unilamellar vesicles; then (b) contacting an organic salt to the unilamellar vesicles; and then (c) detecting a change in at least one thermal parameter of the unilamellar vesicles caused by the organic salt at said known concentration. A change in the at least one thermal parameter indicates the organic salt is potentially toxic for use as a stabilizing agent for live cells or viruses. A further aspect of the invention is a composition comprising, consisting of or consisting essentially of: (a) live cells or viruses; and (b) a stabilization media. The stabilization media comprising, consists of or consists essentially of at least one cryoprotectant, optionally water, an organic salt, (optionally) an inorganic salt, and (optionally) other ingredients such as fetal bovine serum.

Further aspects of the invention include methods of using compositions as described herein for the stabilization or preservation of cells and viruses.

The present invention is explained in greater detail in the drawings herein and the specification set forth below. The disclosures of all US Patent references cited herein are to be incorporated herein by reference in their entirety.

### BRIEF DESCRIPTION OF THE DRAWINGS

FIG. 1: The gel-liquid crystalline transition in a lipid bilayer. (a) Detection of the transition by microcalorimetry. The curve is for a pure dipalmitoylphosphatidylcholine bilayer, and shows a sharp well-defined transition where the area under the curve equals the heat of absorption, ΔH. As the temperature is raised, measurement of the heat absorbed shows a sharp peak at the transition temperature (Tₘ) where the melting of the membrane takes place. (b) Schematic view of melting at the transition temperature. Below this temperature the hydrocarbon tails are packed together in a rigid, nearly solid gel state (left). Above the transition temperature, the chains become free to move about, and the interior of the membrane now adopts a fluid liquid crystalline state (right).

FIG. 2: Representative calorimetric curves for control compound-lipid bilayer mixtures. DSC thermograms of DPPC liposomes in 20 mM HEPES (black), with 10 mM PHCL (gray), with 10 mM NIBU ( [symbol] ), and 10 mM NHCL (⋄). All experiments were done with a 20 mM HEPES background and at pH=7.40.

FIG. 3: Representative calorimetric curves for test compound-lipid bilayer mixtures. DSC thermograms of DPPC liposomes (a) in 20 mM HEPES 20 to 50° C., (b) in 20 mM HEPES 10 to 90° C., (c) 10 mM CBEH in 20 mM HEPES 20 to 50° C., initial scan, (d) 10 mM CBEH in 20 mM HEPES 20 to 50° C., second scan, (e) 10 mM CBEH in 20 mM HEPES 10 to 90° C., initial scan, (f) 10 mM CBEH in 20 mM HEPES 10 to 90° C., second scan. All experiments were done at pH=7.40. Individual plots are not set to the same vertical scale in order to highlight peak trends.

### DETAILED DESCRIPTION OF ILLUSTRATIVE EMBODIMENTS

Cations including organic cations useful for carrying out the present invention are known. In some embodiments, the organic cation is a quaternary ammonium cation such as choline. Additional examples include, but are not limited to, quaternary ammonium compounds that are aliphatic heteroaryl, aliphatic benzylalkyl ammonium, dialphatic dialkyl ammonium, or tetralkyl ammonium compounds. Particular examples of the foregoing and additional examples of suitable cations include but are not limited to those set forth in U.S. Pat. No. 8,232,265 to Rogers et al., the disclosure of which is incorporated herein by reference.

Anions including organic anions useful for carrying out the present invention are known. In some embodiments, the anion is a conjugate base of an organic acid such as a carboxylic acid or sulfonic acid, or an anionic ester of phosphoric acid (organophosphates), citric acid, malic acid, tartaric acid, etc. Particular examples of anions include but are not limited to phosphates (hydrogen phosphate and dihydrogen phosphate), citrate, dihydrogen citrate, hydrogen citrate, saccharinate, gluconate, tartarate, hydrogen tartarate, lactate, formate, levulinate, malate, hydrogen malate, glycolate and lactobionate. Additional examples of suitable anions include but are not limited to those set forth in U.S. Pat. No. 8,232,265 to Rogers et al., the disclosure of which is incorporated herein by reference.

Cryoprotectants useful for carrying out the present invention are known. In some embodiments, the cryoprotectants are alcohols (including sugar alcohols) such as trehalose or sulfoxides such as dimethylsulfoxide (DMSO), including combinations thereof. Particular examples of cryoprotectants include, but are not limited to, acetamide, agarose, alginate, alanine, albumin, ammonium acetate, anti-freeze proteins, butanediol, chondroitin sulfate, chloroform, choline, cyclohexanediols, cyclohexanediones, cyclohexanetriols, dextrans, diethylene glycol, dimethyl acetamide, dimethyl formamide, dimethyl sulfoxide, erythritol, ethanol, ethylene glycol, ethylene glycol monomethyl ether, formamide, glucose, glycerol, glycerophosphate, glyceryl monoacetate, glycine, glycoproteins, hydroxyethyl starch, inositol, lactose, magnesium chloride, magnesium sulfate, maltose, mannitol, mannose, methanol, methoxy propanediol, methyl acetamide, methyl formamide, methyl ureas, methyl glucose, methyl glycerol, phenol, pluronic polyols, polyethylene glycol, polyvinylpyrrolidone, proline, propanediol, pyridine N-oxide, raffinose, ribose, serine, sodium bromide, sodium chloride, sodium iodide, sodium nitrate, sodium nitrite, sodium sulfate, sorbitol, sucrose, triethylene glycol, trimethylamine acetate, urea, valine, xylose, etc., including combinations thereof. See, e.g., U.S. Pat. No. 8,017,311.

“Cells” as used herein, includes both prokaryotic and eukaryotic cells, and both plant cells (including monocots and dicots) and animal cells (e.g., avian, amphibian, reptile, and mammalian cells). In some embodiments mammalian cells (including but not limited to human, monkey, ape, dog, cat, mouse, rat, horse, goat, sheep, etc.) are preferred. Cells may reside in vitro or in vivo in a tissue in organ (though the tissue or organ is generally in vitro rather than in vivo in a host or subject). Additional examples are given below.

“Virus,” as used herein, may be any type of animal or plant virus, including both DNA viruses and RNA viruses. Examples include, but are not limited to, vaccinia virus, modified vaccinia Ankara (MVA) poxvirus, adenovirus, adeno-associated virus, herpesvirus, retroviruses, vesicular stomatitis virus, sendai virus, etc. Additional examples include but are not limited to those viruses set forth in US Patent Application Publication No, 20110000480 to Turner and Ennis, the disclosure of which is incorporated by reference herein in its entirety. The viruses are, in general, live viruses, and in some embodiments may be attenuated viruses. Examples of suitable live attenuated viruses include but are not limited to those set forth in U.S. Pat. No. 8,084,039 to Stinchcomb, the disclosure of which is incorporated herein by reference.

A. Screening methods.

The present invention provides a method of screening an organic salt for potential toxicity if used as a stabilizing agent for live cells or viruses, the method comprises:

(a) providing a composition of unilamellar vesicles, said unilamellar vesicles consisting essentially of dipalmitoylphosphatidylcholine (DPPC); then

(b) contacting an organic salt to said unilamellar vesicles at a known concentration; and then

(c) detecting by differential scanning calorimetry a change in at least one thermal parameter of the unilamellar vesicles caused by said organic salt at said known concentration;

a change in the at least one thermal parameter at a concentration of 1, 2, 10 or 20 mM or less for said organic salt indicating said organic salt is potentially toxic for use as a stabilizing agent for live cells or viruses.

In some embodiments, the change in at least one thermal parameter is: a decrease in the transition peak temperature (Tₘ), an increase in the width of the transition peak at half height (ΔT_{1/2}), a decrease in the enthalpy of the transition change (ΔH), or a combination thereof. A decrease in the transition peak temperature (Tₘ) is particularly preferred for consideration, alone or in combination with other parameters.

While the unilamellar vesicles may be of any suitable size, in some embodiments they are preferably 100 nm in diameter.

While the unilamellar vesicles may be provided in any suitable composition, in some embodiments they are preferably provided as an aqueous dispersion at a pH of 7.4.

The differential scanning calorimetry may be carried out by any suitable technique. In some embodiments, after samples of the aqueous dispersions of vesicles containing the organic salt are loaded into the calorimeter cells, the samples are equilibrated for 15 minutes at 10° C. and then scanned from 20 to 50° C. at 60° C./hour with a 16 second filter and passive feedback (gain).

In some embodiments, the dispersions are allowed to equilibrate overnight at 4-5° C., and then combined with organic salt and used for scanning calorimetry within 16 hours of production (e.g., by extrusion), to limit the inducement of the subgel phase.

### B. Compositions.

As also noted above, a further aspect of the invention is a composition comprising, consisting of or consisting essentially of: (a) live cells or viruses; and (b) a stabilization media. The stabilization media comprising, consists of, or consists essentially of at least one cryoprotectant, optionally water, an organic salt, (optionally) an inorganic salt, and (optionally) other ingredients such as fetal bovine serum.

In some embodiments of the foregoing, the organic salt consists of an organic cation (such as choline) and an inorganic anion (such as dihydrogen phosphate); in other embodiments of the foregoing; the organic salt consists of an organic cation and an organic anion (such as lactate).

In some embodiments, the stabilization media has a total osmolarity of 300, 400 or 500 milliosmoles to 5000, 6000, or 8000 milliosmoles

In some embodiments of the foregoing, the said organic salt including in an amount of from 100, 200, or 300 milliosmoles to 1500, 2000, or 3000 milliosmoles

In some embodiments of the foregoing, the inorganic salt (sometimes a byproduct of the formation of the organic salt, and which itself adds little functionality to the composition) is included in an amount of not more than 500, 600, or 800 milliosmoles.

In some embodiments of the foregoing, the composition has a glass transition temperature (T_{g}) of from −100, −80 or −60° C. up to −30, −20, −10, zero, or 10° C.

Cells that may be included in the composition include, but are not limited to stem cells, germ cells, and differentiated tissue cells. Particular examples include, but are not limited to, nerve cells, skin cells, bone cells, cartilage cells, pancreatic cells, liver cells, artery cells, vein cells, bladder cells, and kidney cells. More particular examples include, but are not limited to, mesenchymal stem cells, amniotic fluid stem cells, adipose stem cells, sperm cells, egg cells, pancreatic islet cells, osteoblasts, osteoclasts, hepatocytes, keratinocytes, chondrocytes, myoblasts, fibroblasts, and peripheral neurons. In some embodiments, the cells are mammalian cells carried in or on a natural or artificial, organic or inorganic, stable or bioerodable, tissue scaffold (e.g., collagen, hydroxyapatite, polylactide, etc.). Particular examples include, but are not limited to, those set forth in U.S. Pat. Nos. 8,278,101, 8,226,715; 8,172,908; 8,137,686; and 8,105,380, the disclosures of which are incorporated herein by reference.

### C. Methods of Use.

A method of stabilizing cells or viruses can be carried out by (a) providing a composition as described above, and then, (b) cooling the composition to a temperature less than the glass transition temperature of the composition and/or drying said composition. In some embodiments, the compositions are simply dried to a glassy state. When the compositions are both cooled and dried, the cooling and drying may be carried out concurrently or in any sequence. Cooling and/or drying may be carried out by any suitable technique, including but not limited to drying, freezing, freeze-drying, vitrification, or combinations thereof. Particular techniques include, but are not limited to, lyophilization, use of a desiccant, microwave drying (including but not limited to the methods described in U.S. Pat. No. 7,883,664), air-flow drying, spin drying, etc., and combinations thereof.

After cooling and/or drying, the compositions may then be stored, typically at a temperature less than the glass transition temperature of the composition, for a desired time (e.g., one or two months, to one or two years, or more). Storage may be in any suitable container or vessel in any suitable device, such as a freezer, refrigerator, liquid nitrogen, etc., depending upon the particular composition.

Following any desired storage, stabilized cells or viruses compositions as described above may be warmed to a temperature above said glass transition temperature and/or rehydrated to provide said cells or viruses in viable form. The cells or viruses may then be used for whatever intended purpose, including but not limited to subsequent in vitro culturing, growing, or propagation thereof, and/or (in the case of tissues or organs for implantation or transplantion) in vivo implantation or transplantation into a subject in need thereof.

The present invention is explained in greater detail in the non-limiting Examples set forth below.

### EXAMPLE 1

### A Screening Assay for Evaluating Toxic Cellular Interactions

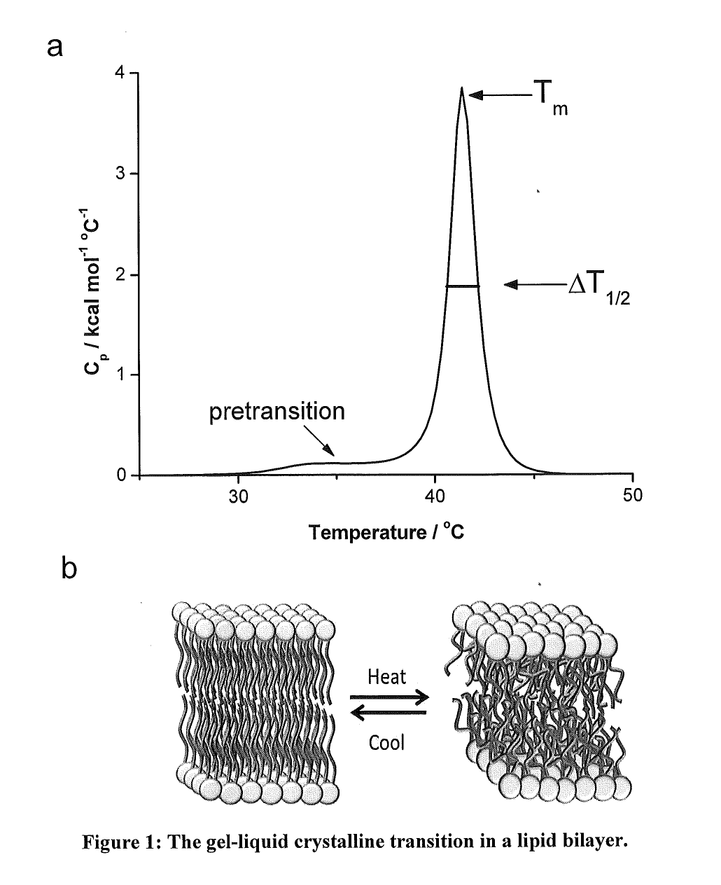

To better understand the nature of specific interactions between ILs and biological interfaces, and the relationship between cytotoxicity and lipophilicity, we investigated the effect of these organic salts on the thermotropic phase behavior of model lipid membrane systems prepared from 1,2-dipalmitoyl-sn-glycero-3-phosphocholine (DPPC). Using calorimetric methods we examined the heat signals associated with the lamellar gel (L_{β}) to lamellar liquid crystalline (L_{α}) phase transition of unilamellar vesicles prepared from DPPC. The temperature at which this event occurs can be described as the melting temperature (Tₘ) of the hydrocarbon chains, as shown in FIG. 1. Because of the tight packing and restricted mobility of hydrocarbon acyl chains in a bilayer arrangement, this transition from the all-trans conformation to the gauche conformation is a highly cooperative event (Taylor, 1995). Molecules that interact or intercalate with the acyl chains can significantly affect the cooperativity of this transition. Thermal parameters that were monitored to probe bilayer effects include the temperature at the transition peak (Tₘ), the width of the transition peak at half height (ΔT_{1/2}), and the enthalpy of the transition change (ΔH) (Demetzos, 2008).

Materials. Lyophilized lipid 1,2-dipalmitoyl-sn-glycero-3-phosphocholine 16:0 (DPPC) was purchased from Avanti Polar Lipids (Alabaster, Ala.). Sodium chloride was obtained from Fisher Scientific (Pittsburgh, Pa.). Crystalline sodium phosphate dibasic, 7-hydrate (Na₂HPO₄.7H₂O) was purchased from J. T. Baker (Phillipsburg, N.J.). Bis(2,4,4-trimethylpentyl)phosphinic acid was obtained from Cytec Industries (Woodland Park, N.J.). Choline chloride, choline hydroxide (20 wt % in H₂O), methanol, dibutyl phosphate, bis(2-ethylhexyl)phosphate, O,O′-diethyl dithiophosphate, nortriptyline hydrochloride, procainamide hydrochloride, ibuprofen sodium salt, and 2-[4-(2-hydroxyethyl)piperazin-1-yl]ethanesulfonic acid (HEPES) were all purchased from Sigma Aldrich (St. Louis, Mo.). All chemicals were used as received, except as otherwise specified. ELGA Purelab Ultra water (18.2 MΩ.cm) was used in the preparation of all water based solutions.

Choline salts. Five choline salts were prepared at Monash University according to previous methods:
(1) 2-hydroxyethyl(trimethyl)azanium dihydrogen phosphate (CDHP, choline dihydrogen phosphate), melting point=195° C., (Vijayaraghavan, 2010),
(2) 2-hydroxyethyl(trimethyl)azanium bis(2,4,4-trimethylpentyl)phosphinate (CTMP, choline bis(2,4,4-trimethylpentyl)phosphinate), melting point=131.2° C. (Weaver, 2010),
(3) 2-hydroxyethyl(trimethyl)azanium O,O-diethyl dithiophosphate (CDEP, choline O,O′-diethyl dithiophosphate), melting point=−43.6° C. (Weaver, 2010),
(4) 2-hydroxyethyl(trimethyl)azanium dibutyl phosphate (CDBP, choline dibutyl phosphate), melting point=79° C. (Fujita, 2007; Weaver, 2010),
(5) 2-hydroxyethyl(trimethyl)azanium bis(2-ethylhexyl)phosphate (CBEH, choline bis(2-ethylhexyl)phosphate), melting point is not well defined, but there is a glass transition (onset temperature)=−83.2° C. (Weaver, 2010),
   CDHP, CTMP, and CDEP are solids at room temperature, but with the addition of small amounts of water (≦20% (w/w)) produce clear liquids. However, they are considered as near-ILs since they are liquid at room temperature when hydrated with 20% (w/w) H₂O) (Hekmat, 2007). CDBP and CBEH are liquid at room temperature.

Preparation of unilamellar vesicles. To study model membranes, unilamellar vesicles were prepared using DPPC. Stock lyophilized DPPC was stored under an argon atmosphere in a −20° C. freezer until used. To prepare lipid solutions, lyophilized DPPC powder was placed into disposable glass culture tubes with two glass beads and 20 mM HEPES buffer at pH 7.40, which had been pre-warmed to 70° C. using a isotemp heating block (Fisher Scientific, Pittsburgh, Pa.). The lipid solutions were then vortexed, and placed in the heating block at 70° C. for 30 minutes with occasional agitation to allow complete hydration. The lipid solutions were then extruded through Nucleopore® track-etched polycarbonate filters (pore size 100 nm) (Whatman, Pleasanton, Calif.) mounted in a mini-extruder (Avanti Polar Lipids, Alabaster, Ala.) fitted with two 0.5 mL Hamilton gas-tight syringes (Avanti Polar Lipids, Alabaster, Ala.). In order to achieve a tighter polydispersity (particle size distribution), the lipid solutions were subjected to 21 passes through the two filters after which the lipid vesicle solutions were collected from the receiving syringe to prevent contamination by particles that had not passed through the filter. The extrusions were refrigerated at 4° C. for a minimum of 24 h before use in calorimetry experiments.

Differential scanning calorimetry. A MicroCal VP-DSC microcalorimeter (MicroCal, Northhampton, Mass.) was used to assess the thermal stability of all samples. DSC baseline repeatability was established with a minimum of 5 reference solution scans. After the refrigerated incubation period described in the previous section, an aliquot of the stock lipid vesicle solution was combined with experimental compounds in 20 mM HEPES buffer at pH 7.40 in a 1:1 ratio by volume where dilution resulted in a 10 mM test compound experimental concentration in the calorimeter cells. The experimental test compound concentration of 10 mM was chosen to allow a comparison of membrane interactions at a level where toxic and non-toxic interactions could be discriminated. (Weaver, 2010). Enough stock lipid vesicle solution was prepared initially to allow a control (lipid only) DSC run for each compound tested. The test solutions were mixed in thermovac tubes (MicroCal, Northhampton, Mass.), and immediately degassed for 5 min using a thermovac vacuum degassing system (MicroCal, Northhampton, Mass.). After degassing, the samples were loaded by syringe into the calorimeter. After loading into the calorimeter cells, the samples were allowed to equilibrate 15 minutes at 10° C. then scanned from 20 to 50° C. at 60° C./hour with a 16 second filter and passive feedback (gain). Reversibility was determined by rescanning samples after allowing them to cool down to 10° C. The samples were repeatedly scanned until at least three consistent thermograms were obtained consecutively. The data was analyzed using Origin software provided by MicroCal in order to permit the determination of the main transition temperature (Tₘ), the main transition full-width-at-half-maximum-height (ΔT_{1/2}), and the calorimetric enthalpy (ΔH). The DPPC dispersions were prepared and allowed to equilibrate overnight at 4-5° C. The dispersions were used within 16 hours of extrusion, and were not incubated longer than overnight in order to limit the inducement of the subgel phase which normally occurs with 4-5 days at 0-4° C. (O'Leary, 1984). Just prior to loading samples into the DSC, the lipid dispersions were gently allowed to warm to room temperature, then mixed with the control compounds (previously prepared in 20 mM HEPES, pH 7.40), and degassed.

Results and discussion. In previous work we have shown that for a series of choline salts ranging from strongly hydrophilic to strongly hydrophobic, the apparent cellular toxicity appeared to trend with the lipophilicity of the compound, and we hypothesized that adverse interactions with the cell membrane were the source of this toxicity (Weaver, 2010). The mammalian cell plasma membrane consists of a variety of biomacromolecules, arranged into complex assemblies that are not readily amenable to calorimetric study. The primary components are proteins and lipids, and simple, single-component bilayer lipid vesicles are often used as model systems for biological cells (Fox, 2006; Fox, 2010; Katz, 2006), To better understand the nature of toxic interactions with cells, model vesicles formed from phospholipids were exposed to choline salts and other well-characterized control compounds to elucidate perturbing effects on the model membranes that might lead to leakiness or general loss of membrane structure. DPPC was chosen as the vesicle forming lipid due to the fact that the major fatty acid in the choline-containing class of glycerophospholipids found in mouse macrophages is palmitic acid (16:0) (Chapkin, 1990). The DPPC model membranes being used in this DSC study are thus intended to serve as the most basic mimics of the mouse macrophage cell line used previously to investigate the relative toxicity of these same organic salts.

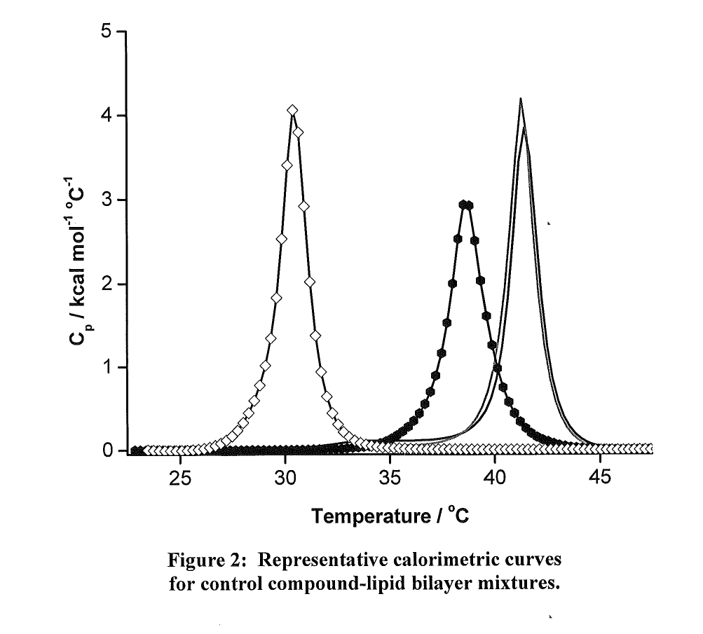

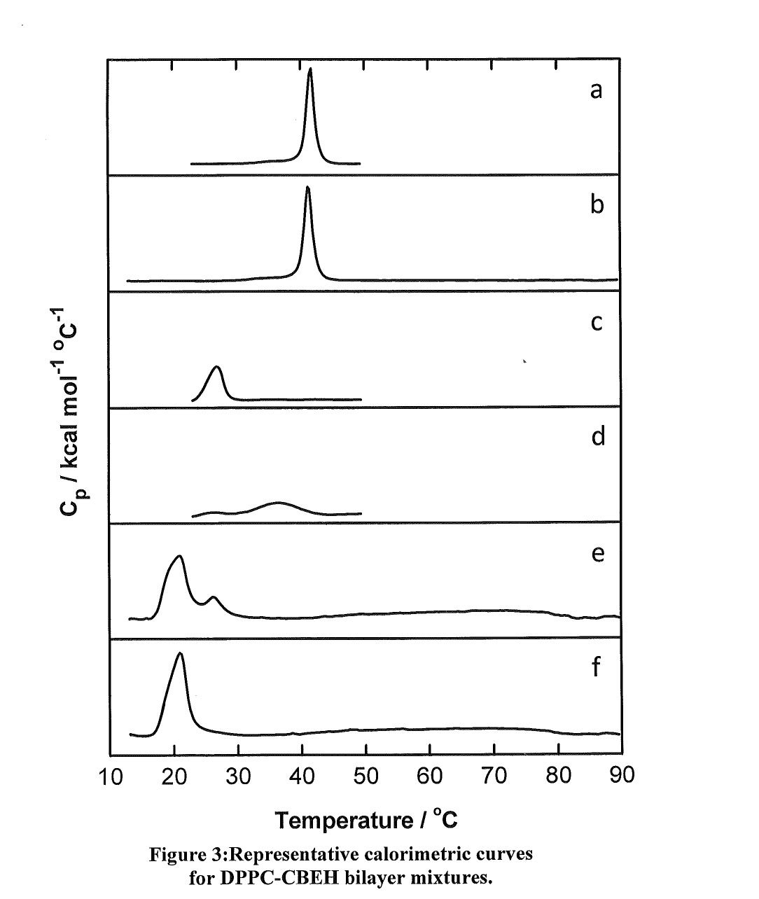

In order to identify the specific thermal signatures associated with different types of interfacial interactions, control compounds with well-known effects on cell membranes were added to DPPC vesicles, and changes in the membrane transition behavior of the lipids were evaluated using DSC. DSC measures enthalpy changes associated with a given phase change, and representative DSC curves from 20 to 50° C. for the control compounds are illustrated in FIG. 2. The endotherms for DPPC vesicles in 0.20 mM HEPES at pH 7.40 without added control compounds exhibited the characteristic thermotropic behavior, demonstrating both a pretransition at 34° C. and a gel-liquid crystal phase transition at 41.4° C., in agreement with previous reports (Levin, 1981). Mixtures of choline salts and DPPC vesicles were studied at similar lipid concentrations (approximately 0.39 mM) under the same experimental conditions to allow comparison of specific interactions between the salts and the lipid vesicles. Simple physiological salts were also included for comparison. In order to compare the thermotropic behavior of DPPC in relation to each compound, the main transition temperature, the enthalpy change, and the level of cooperativity (ΔT_{1/2}) with respect to each compound were gathered for comparison in Table 1, along with indications regarding pretransition effects. Previously published EC₅₀ (mM) values for the set of choline salts investigated here are reproduced in Table 1 where the compounds are ranked based on their relative cellular toxicity. Initial heating scans were also compared with subsequent reheating scans of the same sample. Representative scans are shown in FIG. 3.

Low toxicity salts. As shown in Table 1, CDHP and CDBP had no statistically significant effect on the DPPC main gel-liquid crystal phase transition (ΔTₘ, ΔΔH, and ΔΔT_{1/2}), suggesting that there was no interaction between these salts and the acyl chains of the lipids at the studied concentration. The most benign salts tested previously for cytotoxicity were NaCl and choline chloride (CCl) with EC₅₀ₛ of 63 and 34 mM respectively (Table 1) (Weaver, 2010). The thermotropic behavior of these two salts was comparable to the low toxicity choline compounds, as well as the control compound procainamide, all having a little to no effect on the main transition enthalpy and little effect on Tₘ and cooperativity (ΔΔT_{1/2})compared to control DPPC runs from the same lipid batch solutions (Table 1). Procainamide has previously been classified as nonmembrane active towards DMPC (Katz, 2006), and appears to have the same effect on DPPC. This suggests that the variation in toxicity observed with these compounds (CDHP: EC₅₀=20 mM and CDBP: EC₅₀=9 mM) when compared to more cellularly benign NaCl (EC₅₀=60 mM) might have more to do with an aspect of cellular function, such as alteration of membrane proteins, ionic strength effects, nutrient sequestration or metal chelation, and does not appear to be due to lipid membrane localization (Table 1).

It was noted that the pretransition peak normally seen at approximately 34° C. was not resolvable in the thermal scans of DPPC in the presence of CDEP (EC₅₀=8 mM) while the main transition demonstrated significant broadening indicating a less cooperative gel-liquid crystal phase transition (Table 1). This might suggest that CDEP is interacting with the surface of the lipid bilayer in a manner similar to that of nortriptyline. With the addition of nortriptyline to DPPC, the thermal transitions were shifted to lower temperatures, with notable main peak broadening (ΔΔT_{1/2}) indicating reduction in the molecular ordering of lipid molecules within the vesicle bilayer and reduction in the cooperativity of melting without altering the overall domain organization to such an extent that overall enthalpy was changed (Katz, 2006; Fox 2010). The magnitude of these changes in the thermal trace can vary considerably depending on the extent of molecular re-arrangement at the surface. A confocal Raman spectroscopy investigation on the partitioning of nortriptyline into DPPC vesicles indicated that this molecule is located in the lipid interfacial/headgroup region with the cyclic rings of the structure associating near the acyl chains with the polar carbon tail “snorkeling” into the lipid headgroup (Fox, 2009; Katz, 2006). (data not shown). Head group interactions of this nature can cause a significant depression in the transition temperature of the gel-liquid crystalline phase change (Fox, 2010). This type of interaction with a lipid bilayer would probably result in changes in membrane permeability that could lead to adverse cellular interactions, contributing to increased toxicity of this compound compared to the other salts in this category. In the case of CDEP this effect appears to be moderate, but could account for the slightly lower EC50 value compared to CDHP and CDBP.

High toxicity salts. The organic salts CBEH and CTMP were both observed to have a high toxicity in previous studies. Their structures suggest that they would be highly lipophilic, and thus likely to cause perturbations in membrane structure that would affect lipid phase transitions. Changes in lipid transition behavior were indeed observed. The salt CTMP (EC₅₀=<0.25 mM) reduced the main transition Tₘ by 21% to 32.6° C. compared to that for DPPC alone (Table 1), reduced the transition enthalpy by half, and increased the cooperativity of the transition by decreasing the peak width (ΔΔT_{1/2}) by 17% compared to control lipid vesicles run from the same stock batch solution (FIG. 3a, Table 1). CTMP also reduced the pretansition peak below resolvable limits. Clearly there were significant interactions with the lipid membrane, consistent with both acyl chain interaction and head group interaction. Intercalation of CTMP into the lipid membrane acyl chain region would give a signature much like the control compound ibuprofen, i.e., a lower Tₘ and reduction in enthalpy. Typically, acyl chain interactions are not associated with a change in the pretransition temperature or enthalpy, but it is possible that the temperature associated with pretansition peak has been reduced as well, moving below the range tested (20 to 50° C.). The branched structure of CTMP would suggest that if the compound penetrated into the acyl chains, considerable disorder would be expected. It is possible that the acyl chain disordering is so extensive that the headgroup region is altered as a secondary effect, and not necessarily because of a direct surface interaction with CTMP. This hypothesis would explain why the thermal signature displays characteristics of both strong acyl chain interactions (depressed main transition temperature and enthalpy) and molecular changes around the head group (loss of the pre-transition peak).

The last choline salt investigated, CBEH, which has a branched alkyl chain anionic structure and an EC₅₀ value <0.30 mM, exhibited much more complex thermotropic behaviour than the other compounds and appeared to influence the tendancy toward lipid polymorphism. For each of the compounds investigated, including the controls, thermal reversibility was tracked by allowing the samples to cool back to 20° C. following the initial heating scan and then reheating for at least 5 scans. For all of the compounds tested, except CBEH, the gel-liquid crystal phase transition was reversible with less than a 5% reduction in enthalpy upon reheating (data not shown). The initial heating scan of DPPC in the presence of CBEH underwent a barely detectable pretransition near 33° C., and a main transition near 41° C., with a reduction in enthalpy of 82 and 99%, respectively, compared to DPPC in HEPES alone (FIG. 3b, Table 1). However, a third prominent peak was observed at 27° C. Others have reported a DPPC subgel crystalline bilayer (L_{c}) state at this temperature, depending on how the lipid was rehydrated (Meyer, 2000). That is, in the presence of CBEH, DPPC underwent three phase transitions with four possible lamellar phases observed across the temperature scan. The peak observed at 20° C. is suspected to be associated with the crystalline (subgel) bilayer (L_{c}) phase, followed by a subtransition at 27° C. into the gel phase (L_{β′}), a pretransition at 36° C. into the ripple phase (P_{β′}), and finally the main transition at 42° C. into a liquid-crystalline bilayer (L_{α}) phase, each close to that observed for DPPC in water previously (FIG. 3b) (Levin, 1981; Meyer, 2000). The significance imparts from the fact that the DPPC batch solution was incubated for the same temperature and time period as all the other batch solutions, and there was no subgel phase observed in the presence of any of the other compounds. Further, only the first heating scan yielded these results. The second through fourth reheating scans after cooling back to 20° C. resulted in a reversible but altered dispersion where a subgel phase transition with a much reduced enthalpy occurred, followed by a pretransition of greater enthalpy without a resolvable main transition It is possible that there was a uniform population of vesicles that underwent all of these transitions in the first scan, but more likely there was a mixed populations of vesicles present during the first scan, that ultimately converged to a uniform population after repeated thermal scanning.

The more toxic compounds (CTMP and CBEH) clearly altered the observed lipid phases of DPPC by reducing the Tₘ and enthalpy of the main transition or by enhancing the equilibration of alternate phases. The EC₅₀s previously reported for CTMP and CBEH were upper limits of cytotoxicity, therefore it is possible that these compounds have different effects on lipid membranes that might result in some differentiation, especially if they were evaluated at lower levels of exposure. CTMP has two fewer oxygens bound to the central anionic phosphorous atom, and is a phosphinate unlike the other compounds tested, which all carry a phosphate anion. (data not shown). Both the TMP and BEH anions are similar in structure in that both have branched alkyl chains as substituents (Table 1). It is possible that CBEH is causing a degree of lipid polymorphism that would play a critical role in the cytotoxicity mechanism due to its potential influence on macrophage membrane structure (Weaver, 2010). The chain-melting transition of DPPC in the presence of CTMP observed at 33° C. is fully reversible with all subsequent scans comparable to the first heating scan. With subsequent rescanning across the same temperature range, a new lipid structure appears that has different chain packing and undergoes fully reversible transitions of much lower enthalpy with no evidence of the reappearance of the transition at 27° C. With the loss of the apparent subtransition located at 27° C., the total enthalpy associated with all three transitions was reduced as well. To determine if this were a true loss in lipid ordered structure or a conversion to another structure (as with polymorphism) within the temperature range investigated, additional dispersions of DPPC-CBEH were run starting at a lower temperature (10° C.). In the first heating scan a new peak was found with a Tₘ=21° C. with approximately double the enthalpy associated with the peak located at 27° C., and in the second heating scan, the transition located at 27° C. again dissipated as before with a concomitant increase in enthalpy of the transition located at 21° C. (data not shown). This transition has been noted in cold-hydrated DPPC dispersions (hydration and storage at 4° C.), but it was unexpected in these warm-hydrated dispersions (hydration above 50° C. and storage at 4° C.). The presence of this second type of subgel (crystal) phase has been associated with lamellae that have convex-concave (egg-carton) bilayer deformations and curvature that is considered “soft” rather than crystalline in configuration. This subtransition is distinguished from the subgel L_{c} phase and designated as the subgel P_{cc} phase (Meyer, 2000). If CBEH favors the development of a “soft” DPPC lamellae structure at physiologically temperatures, whereas CTMP does not, then this would be an additional contribution to the cytotoxicity of this compound, likely inducing a direct injury that would result in dissociation of the cell membrane, compared to a disruption in the membrane structure as we might expect with CTMP.

TABLE 1
Thermodynamic parameters of the phase transitions undergone by 1,2-dipalmitoyl-
sn-glycero-3-phosphocholine 16:0 PC (DPPC) in different liposome preparations ^{a}

| | | | | | | | | Change in | |
|---|---|---|---|---|---|---|---|---|---|
| | Main Transition | | | Change in Main Transition ^{d} | | | Pretransition | Pretransition ^{d} | |
| | Tₘ | ΔH | ΔT_{(1/2)} | ΔTₘ | ΔΔH | ΔΔT_{(1/2)} | ΔH | ΔH | EC₅₀ ^{e} |
| Sample ^{b, c} | (° C.) | (cal mol⁻¹) | (° C.) | (° C.) | (cal mol⁻¹) | (° C.) | (cal mol⁻¹) | (cal mol⁻¹) | (mM) |

Control Compounds

| | | | | | | | | | |
|---|---|---|---|---|---|---|---|---|---|
| Nortriptyline | 30.66 | 8078 | 1.41 | −26% | +8% | +6% | NR | −100% | — |
| hydrochloride | (0.05) | (78) | (0.04) | | | | | | |
| Procainamide | 41.46 | 7892 | 1.44 | +0.2% | +0.3% | −9% | 173 | −47% | — |
| hydrochloride | (0.01) | (209) | (0.03) | | | | (13) | | |
| Ibuprofen | 38.82 | 7222 | 1.79 | −6% | −11% | +12% | 375 | −13% | — |
| sodium salt | (0.15) | (170) | (0.04) | | | | (47) | | |

Choline Salts

| | | | | | | | | | |
|---|---|---|---|---|---|---|---|---|---|
| CDHP | 41.69 | 7949 | 1.33 | +0.7% | +3% | −6% | 248 | −54% | 20 |
| | (0.01) | (121) | (0.00) | | | | (14) | | |
| CDBP | 41.05 | 7831 | 1.50 | −0.5% | +2% | −6% | 440 | −20% | 9.1 |
| | (0.13) | (218) | (0.09) | | | | (73) | | |
| CDEP | 40.68 | 8102 | 1.71 | −2% | +2% | +14% | NR | −100% | 8.2 |
| | (0.02) | (53) | (0.04) | | | | | | |
| CBEH ^{f} | 42.49 | 77 | 2.95 | +2.5% | −99% | +16% | 66 | −82% | <0.30 |
| | (1.21) | (>23) | (>0.90) | | | | (>20) | | |
| CTMP ^{g} | 32.58 | 3580 | 1.48 | −21% | −52% | −17% | NR | −100% | <0.25 |
| | (0.09) | (11) | (0.00) | | | | | | |

Inorganic/High Melting Point Organic Salts

| | | | | | | | | | |
|---|---|---|---|---|---|---|---|---|---|
| NaCl | 41.72 | 9823 | 1.53 | +0.8% | +17% | −4% | 508 | +103% | 63 |
| | (0.03) | (134) | (0.06) | | | | (38) | | |
| CCL | 41.70 | 9633 | 1.55 | +0.8% | +15% | −3% | 378 | +51% | 34 |
| | (0.02) | (133) | (0.04) | | | | (7) | | |

^{a} All samples were prepared in 0.20 mM HEPES buffer at pH 7.40. All compound concentrations are 10 mM. A control DPPC scan was obtained on the stock LUVs batch solutions as prepared for all compounds tested. Triplicate testing was run for all compounds except where otherwise noted. Values reported are for equilibrium scans which were reproducible after rescanning for a minimum of four scans unless otherwise noted. Not resolvable (NR) under these experimental conditions. The standard error is given in parenthesis.
^{b} CDHP, choline dihydrogen phosphate; CTMP, choline bis(2,4,4-trimethylpentyl)phosphinate; CDEP, choline O,O′-diethyl dithiophosphate; CDBP, choline dibutyl phosphate; CBEH, choline bis(2-ethylhexyl)phosphate; CCL, choline chloride; NACL, sodium chloride.
^{c} Grand average from all DPPC control runs combined: Main Transition Tₘ = 41.35 ± 0.05° C., ΔH = 7892 ± 100 cal mol⁻¹, ΔT_{1/2} = 1.62 ± 0.06° C.; Pretransition ΔH = 364 ± 26 cal mol⁻¹, n = 14.
^{d} Change in thermodynamic parameters induced by compound compared to DPPC control scan from same LUV batch solution.
^{e} EC₅₀ values reproduced from Weaver et al. (2010).
^{f} The gel-to-liquid-crystalline transition is not reversible, i.e., there is not an observable main transition after cooling and rescanning in the presence of this compound. The resolution of the pretransition and main transition peaks is low, and there is a higher degree of error in the reported the calculated enthalpies.
^{g} Average values from two replicates.

### EXAMPLE 2

### Toxicity Screening of Additional Choline Salts

Microcalorimetric observation of the gel to liquid crystalline transition in small unilamellar vesicles of dipalmitoyl sn glycero phosphocholine (DPPC) treated with various choline salts at a concentration of 10 mM in the assay system described above was carried out. Results are given in Table 2 below.

TABLE 2
Screening of choline salts in DPPC vesicle assay.

| | Δ Tₘ | ΔΔ T_{(1/2)} | ΔΔ H | Log | Log EC₅₀ |
|---|---|---|---|---|---|
| Anion | (C) | (C) | (cal/mole/ C) | Pow | (uM) |
| Dihydrogen | +0.52 | 0.131 | −76 | −3.15 | 4.31 |
| Phosphate | | | | | |
| Tartarate | +0.35 | −0.03 | +121.9 | 03.04 | — |
| Lactate | +0.37 | −0.06 | −480 | −1.73 | — |
| Formate | +0.31 | −0.02 | −733 | −1.53 | — |
| Levulinate | −0.21 | 0.39 | −2155 | −1.18 | — |
| Bis (2,2,4− | −8.08 | −0.37 | −2011 | 3.60 | 2.39 |
| trimethylpentyl) | | | | | |
| Phosphinate (BTP) | | | | | |

For BTP, A large decrease in Tₘ and enthalpy, and increased cooperativity, was observed. For levulinate, a moderate decrease in Tₘ, and a large decrease in enthalpy, was observed. For lactate and formate, a moderate increase in Tₘ and a moderate decrease in enthalpy was observed.

Among other things, these date indicate that choline dihydrogen phosphate, choline tartarate, choline lactate, and choline formate, may be potentially useful organic salts for use in stabilizing live cells and viruses.

### REFERENCES

Aldridge, S., Biobanking emerging as a key growth area, Genet. Engineer. News 25 (3) (2005)).

Baust, G., A Crisis in Biobanking, Cell Preservation Tech 3, 1-2 (2005)

Chapkin, R. S., Carmichael, S. L., Effect of dietary blackcurrant seed oil on mouse macrophage subclass of choline and ethanolamine glycerophospholipids, J. Nutr., 1990, 120(8), 825-830.

Demetzos, C., Differential scanning calorimetry (DSC): A tool to study the thermal behavior of lipid bilayers and liposomal stability, J. Lipo. Res., 2008, 8, 159-173.

Elliott, G. et al, Cell Preservation Tech. 6, 377-384 (2008)).

Foureau, D. M., Vrikkis, R. M., Jones, C. P., Weaver, K. D., MacFarlane, D. R., Salo, J. C., McKillop, I. H., Elliott, G. D., In Vitro assessment of choline dihydrogen phosphate (CDHP) as a vehicle for recombinant human interleukin-2 (rhIL-2), Cell. Mol. Bioeng., 2012, in press.

Fox, C. B., Harris, J. M., Confocal Raman microscopy for simultaneous monitoring of partitioning and disordering of tricyclic antidepressants in phospholipid vesicle membranes, J. Raman Spectrosc. 2010, 41, 498-507.

Fox, C. B., Horton, R. A., Harris, J. M., Detection of drug-membrane interactions in individual phospholipid vesicles by confocal Raman microscopy, Anal. Chem., 2006, 78, 4918-4924.

Fujita, K., MacFarlane, D. R., Forsyth, M., Yoshizawa-Fujita, M., Murata, K., Nakamura, N., Ohno, H., Solubility and stability of cytochrome c in hydrated ionic liquids: Effect of oxo acid residues and kosmotropicity, Biomacromolecules, 2007, 8, 2080-2086.

Fujita, K., Forsyth, M., MacFarlane, D. R., Reid, R. W., Elliott, G. D., Unexpected Improvement in Stability and Utility of Cytochrome c by Solution in Biocompatible Ionic Liquids, Biotechnol. Bioeng. 2006, 94, 1209-1213.

Fujita, K., MacFarlane, D. R., Forsyth, M., Protein solubilising and stabilising ionic liquids, Chemical communications (Cambridge, England), 2005, 4804-4806.

Hekmat, D., Hebel, D., Joswig, S., Schmidt, M., Weuster-Botz, D., Advanced protein crystallization using water-soluble ionic liquids as crystallization additives, Biotechnol. Lett. 2007, 29, 1703-1711.

Jastorff, B., Mölter, K., Behrend, P., Bottin-Weber, U., Filser, J., Heimers, A., Ondruschka, B., Ranke, J., Schaefer, M., Schröder, H., Stark, A., Stepnowski, P., Stock, F., Störmann, R., Stolle, S., Welz-Biennann, U., Ziegerta S., Thöminga, J., Progress in evaluation of risk potential of ionic liquids—basis for an eco-design of sustainable products, Green Chemistry, 2005, 7, 362-372.

Jastorff, B., Störmann, R., Ranke, J., Mölter, K., Stock, F., Oberheitmann, B., Hoffmann, W., Hoffmann, J., Nüchter, M., Ondruschka B., Filser, J., How hazardous are ionic liquids? Structure—activity relationships and biological testing as important elements for sustainability evaluation, Green Chemistry, 2003, 5, 136-142.

Katkov, I. et al., Int. J. of Ref. 29, 346-357 (2006)).

Katz, M., Ben-Shlush, I., Kolusheva, S., Jelinek, R., Rapid colorimetric screening of drug interaction and penetration through lipid barriers, Pharmaceutical Research, 2006, 23(3), 580-588.

Levin, I. W., Bush, S. F., Evidence for acyl chain trans/gauche isomerization during the thermal pretransition of dipalmitoyl phosphatidylcholine bilayer dispersions, BBA—Biomembranes, 1981, 640, 760-766.

Meyer, H. W., Semmler, K., Rettig, W., Pohle, W., Ulrich A. S., Grage, S., Selle, C., Quinn, P. J., Hydration of DMPC and DPPC at 4° C. produces a novel subgel phase with convex-concave bilayer curvatures, Chem. Phys. Lipids, 2000, 105, 149-166.

Mills, F., The Need for Good Storage Practice, Biopreservation and Biobanking 8, 115-117 (2009)).

O'Leary, T. J., Levin, I. W., Effects of solvent on biomembrane structure: Raman spectroscopic investigation of dipalmitoylphosphatidylcholine dispersed in N-ethylammonium nitrate, J. Phys. Chem., 1984, 88, 4074-4078.

Petkovic, M., Ferguson, J. L., Gunaratne, H. Q. N., Ferreira, R., Leitão, M. C., Seddon, K. R., Rebelo, L. P. N., Pereira, C. S., Novel biocompatible cholinium-based ionic liquids: toxicity and biodegradability, Green Chem., 2010, 12, 643-649.

Ranke, J., Mölter, K., Stock, F., Bottin-Weber, U., Poczobutt, J., Hoffmann, J., Ondruschka, B., Filser, J., Jastorff, B., Biological effects of imidazolium ionic liquids with varying chain lengths in acute Vibrio fischeri and WST-1 cell viability assays, Ecotoxicology and Environmental Safety, 2004, 58, 396-404.

Sitaula, R. et al., Biopreservation and Biobanking 7, 95-106 (2009)).

Stolte, S., Matzke, M., Arning, J., Böschen, A., Pitner, W.-R., Welz-Biermann, U., Jastorff B., Ranke, J., Effects of different head groups and functionalised side chains on the aquatic toxicity of ionic liquids, Green Chemistry, 2007, 9, 1170-1179.

Stolte, S., Arning, J., Bottin-Weber, U., Matzke, M., Stock, F., Thiele, K., Uerdingen, M., Welz-Biermann, U., Jastorff B., Ranke, J., Anion effects on the cytotoxicity of ionic liquids, Green Chemistry, 2006, 8, 621-629.

Taylor, K. M. G,, Morris, R. M., Thermal analysis of phase transition behavior in liposomes, Thermochimica Acta, 1995, 248, 289-301.

Vijayaraghavan, R., Thompson, B. C., MacFarlane, D. R., Kumar, R., Surianarayanan, M., Aishwarya, S., Sehgal, P. K., Biocompatibility of choline salts as crosslinking agents for collagen based biomaterials, Chem. Commun., 2010, 46, 294-296.

Vrikkis, R. M., Fraser, K. J., Fujita, K., MacFarlane, D. R., Elliott, G. D., Biocompatible ionic liquids: A new approach for stabilizing proteins in liquid formulation, J. Biomechan. Eng. 2009, 131, 074514.

Weaver, K. D., Kim, H.-J., Sun, J., MacFarlane, D. R., Elliott, G. D., Cyto-toxicity and bio compatibility of a family of choline phosphate ionic liquids designed for pharmaceutical applications, Green Chem., 2010, 12, 507-513.

The foregoing is illustrative of the present invention, and is not to be construed as limiting thereof. The invention is defined by the following claims, with equivalents thereof to be included therein.

That which is claimed is:

1. A method of screening an organic salt for potential toxicity if used as a stabilizing agent for live cells or viruses, the method comprising:
(a) providing a composition of unilamellar vesicles, said unilamellar vesicles consisting essentially of dipalmitoylphosphatidylcholine (DPPC); then
(b) contacting an organic salt to said unilamellar vesicles at a known concentration; and then
(c) detecting by differential scanning calorimetry a change in at least one thermal parameter of said unilamellar vesicles caused by said organic salt at said known concentration;
a change in said at least one thermal parameter at a concentration of 10 mM or less for said organic salt indicating said organic salt is potentially toxic for use as a stabilizing agent for live cells or viruses.

2. The method of claim 1, wherein said change in at least one thermal parameter is selected from the group consisting of a decrease in the transition peak temperature (Tₘ), an increase in the width of the transition peak at half height (ΔT_{1/2}), a decrease in the enthalpy of the transition change (ΔH), and combinations thereof.

3. A composition, comprising:
(a) live cells or viruses; and
(b) a stabilization media, said stabilization media comprising, at least one cryoprotectant, optionally water, optionally fetal bovine serum, an organic salt, and optionally an inorganic salt;
said organic salt consisting of an organic cation and an anion, said anion selected from the group consisting of phosphate anions and organic anions;
said stabilization media having a total osmolarity of 300 to 5000 milliosmoles; with said organic salt including in an amount of from 100 to 1500 milliosmoles; and with said inorganic salt included in an amount of not more than 500 milliosmoles;
said composition having a glass transition temperature (T_{g}) of from −80° C. to −10° C.

4. The composition of claim 3, wherein said organic salt decreases the Tₘ of unilamellar vesicles consisting essentially of dipalmitoylphosphatidylcholine (DPPC) when contacted to said vesicles at a concentration of 10 mM or less, as determined by differential scanning calorimetry.

5. The composition of claim 3, wherein said organic cation is a quaternary ammonium cation.

6. The composition of claim 3, wherein said organic cation is choline.

7. The composition of claim 3, wherein said anion is a conjugate base of an organic acid selected from the group consisting of carboxylic acids, sulfonic acids, and anionic esters of phosphoric acid (organophosphates) citric acid, malic acid and tartaric acid

8. The composition of claim 3, wherein said anion is selected from the group consisting of phosphates (hydrogen phosphate and dihydrogen phosphate), citrate, dihydrogen citrate, hydrogen citrate, saccharinate, gluconate, tartarate, hydrogen tartarate, lactate, formate, levulinate, malate, hydrogen malate, glycolate and lactobionate.

9. The composition of claim 3, wherein said at least one cryoprotectant is selected from the group consisting of alcohols, sulfoxides, and combinations thereof.

10. The composition of claim 3, wherein said at least one cryoprotectant comprises trehalose.

11. The composition of claim 3, wherein said cells or viruses are selected from the group consisting of stem cells, germ cells, and differentiated tissue cells.

12. The composition of claim 3, wherein said cells or viruses are differentiated tissue cells selected from the group consisting of nerve cells, skin cells, bone cells, cartilage cells, pancreatic cells, liver cells, artery cells, vein cells, bladder cells, and kidney cells.

13. The composition of claim 3, wherein said cells or viruses are selected from the group consisting of mesenchymal stem cells, amniotic fluid stem cells, adipose stem cells, sperm cells, egg cells, pancreatic islet cells, osteoblasts, osteoclasts, hepatocytes, keratinocytes, chondrocytes, myoblasts, fibroblasts, and peripheral neurons.

14. The composition of claim 3, wherein said cells or viruses are mammalian cells carried in or on a natural or artificial tissue scaffold.

15. A method of stabilizing cells or viruses, comprising:
(a) providing a composition of claim 3;
(b) cooling said composition to a temperature less than said glass transition temperature of said composition and/or drying said composition.

16. The method of claim 15, further comprising the step of:
(d) storing said composition at a temperature less than said glass transition temperature of said composition for a time of from 1 month to two years.

17. The method of claim 15, wherein said step (b) is carried out by drying, freezing, freeze-drying, vitrification, or combinations thereof.

18. A method of reconstituting stabilized cells or viruses to viable form, comprising:
(a) providing a composition of claim 3 stored at a temperature less than the glass transition temperature of said composition; and then
(b) warming said composition to a temperature above said glass transition temperature and/or rehydrating said composition to provide said cells or viruses in viable form.

19. The method of claim 18, wherein said cells or viruses have been stored at a temperature less than said glass transition temperature for a time of from 1 month to two years.

20. The method of claim 16, further comprising the steps of culturing, growing or propagating said cells or viruses in vitro or in vivo.
